(19) United States
(12) Patent Application Publication
(10) Pub. No.: US 2007/0212342 A1
(43) Pub. Date: Sep. 13, 2007

(54) Protease compositions for the treatment of damaged tissue

(75) Inventors: William O. Kling, Dallas, TX (US); Laura K.S. Parnell, Missouri City, TX (US); Philip J. O'Neill, Bennington, VT (US)

Correspondence Address: MORGAN LEWIS & BOCKIUS LLP, 1111 PENNSYLVANIA AVENUE NW, WASHINGTON, DC, 20004, US

(73) Assignee: Swiss-American Products, Inc.

(21) Appl. No.: 11/642,274

(22) Filed: Dec. 19, 2006

Related U.S. Application Data
(60) Provisional application No. 60/752,288, filed on Dec. 20, 2005.

(51) Int. Cl. A61K 38/48

(52) U.S. Cl. 424/94.63

(57) ABSTRACT

The invention is directed to compositions containing one or more proteases that are beneficial in the treatment of damaged tissue, wounds and inflammation. The compositions of the invention modulate the levels and activity of proteins that are present at wound and inflammation sites and promote the repair of damaged tissue.

### CROSS REFERENCES TO RELATED APPLICATIONS

This application claims the benefit of U.S. Provisional Application No. 60/752,288, filed Dec. 20, 2005.

### BACKGROUND OF THE INVENTION 1. Technical Field of the Invention

The present invention relates generally to the modulation of the protein profile within a body's tissue or its surrounding environment. The invention also relates to the field of wound healing and treatment of damaged tissue conditions and symptoms of disease such as inflammation.

2. Description of the Prior Art

Humans are capable of replacing injured skin and cells by repairing tissue damage. Typically the defect is initially replaced by a fibrous scar, which is later remodeled. During the transitional coagulation stage there is temporary wound closure through the formation of a blood clot consisting of thrombocytes and fibrin. The a-granules in the thrombocytes release various growth factors such as PDGF, IGF-I, TGF-α and EGF. TGF-α and tumor necrosis factor (TNFα) are secreted from vascular endothelial cells, keratinocytes and fibroblasts inducing the inflammatory stage. This stage lasts only a few days under normal conditions. Granulocytes and macrophages that are present in the wound continuously produce cytokines and proteases which degrade injured or denatured extracellular matrix (ECM). Macrophages continue secreting inflammatory and pro-inflammatory cytokines maintaining the inflammatory response until down-regulation and movement into the next stage of healing occurs. In wounds with intact skin, but having underlying tissue trauma such as sports injury or hematoma, although the skin is not replaced, the body nevertheless undergoes an inflammatory response and must remove dead or injured tissue and cells.

Following the inflammatory stage, vascular angiogenesis with capillary formation and development of granulation tissue occurs during the subsequent granulation stage. In this stage, predominantly collagen replaces the basic matrix made up of fibrin, fibronectin and hyaluronic acid.

Common characteristics of all healing types are the consecutive closure of the wound and the simultaneous replacement of the injured tissues. While most wound portions are filled by connective cell material some tissues such as brain, nerves, connective tissue and bones are replaced by other appropriate and adequate material.

Wound healing is a complicated process. Acute wounds are those that heal rapidly and proceed through the inflammatory, proliferation and remodeling phases of wound healing. However, chronic wounds often become senescent in the inflammatory or proliferation stages and cannot progress to closure. In addition to implementing treatment regimens that address the etiology and symptoms, clinicians prepare the wound for healing by removing dead tissue, reducing the bacterial bioburden, decreasing edema, managing exudate, and enhancing angiogenesis. But even though the wound bed may appear ready to heal, the microenvironment may be out of balance thus impeding healing and frustrating both the patient and the clinician.

The microenvironment of the wound is a web of intertwining, cells, proteins, enzymes, fluids, and pathways, which perform specific functions that normally are tightly regulated. In wounds that chronically fail to heal, however, the microenvironment has become deregulated with key components being over-expressed, under-expressed, inactive, or ineffective. Specific protein comparisons between acute and chronic wounds revealed, chronic wounds generally have excessive levels of matrix metalloproteinases (MMPs), high levels of inflammatory cytokines TNFα, IL-1 and IL-6, and minimal levels of tissue inhibitor metalloprotainases (TIMPs) and growth factors like TGFβ, and EGF. To complicate matters, activated inflammatory cells stimulate MMP production and suppress TIMPs by secreting TNFα and IL1-β, which impair the healing process via increased inflammation and degradation of ECM components, growth factors, and receptors contributing to multiple negative feedback loops preventing wound closure.

Promoting, returning to, and maintaining a normal wound microenvironment can be difficult task. Past use of isolated molecules or compounds to modify the healing process has been met with limited success. These limitations may be due to one molecule trying to modify the entire wound environment in a narrowly selected function or due to the duplicity of multiple alternative pathways, or both. Additionally, the hostile chronic wound environment probably degrades exogenously applied growth factors as easily as the intrinsic ones, resulting in little clinical or molecular impact.

An alternative way to return to a more normal wound microenvironment is to modulate the activity of proteins such as MMPs and pro-inflammatory cytokines, which help promote the hostile environment when in excess. MMPs are normally prevented from destroying too much extracellular matrix (ECM) and tissue by the action of TIMPs that form very specific inhibitory complexes with the MMPs. However, in chronic wounds the ratio of MMP to TIMP is high, such that most of the MMPs are uninhibited. In fact, with elevated MMP levels, the TIMP molecules themselves can be hydrolyzed.

Hence, additional approaches are needed to modulate the action and levels of specific proteins in order to promote tissue repair and wound healing, as well as to improve the overall environment of a tissue and its surroundings during the healing process.

### SUMMARY OF THE INVENTION

The invention is directed to methods for modulating the protein profile of a tissue or its surrounding environment to promote the repair of a damaged tissue, or one that is otherwise compromised by disease or injury, by administering a composition containing proteases is administered to the affected area.

The invention is directed to compositions that comprise at least one protease; and optionally a pharmaceutically acceptable carrier, diluent or excipient, wherein the protease can modulates the action or level of at least one protein, wherein said protein is a wound-related protein or an inflammation-related protein.

The invention is also directed to the use of a composition that contains at least one protease, in the manufacture of a pharmaceutical to treat damaged tissue.

The invention is further directed to a method of therapy where a composition containing proteases is administered to a subject in an amount to treat damaged tissue.

### DETAILED DESCRIPTION OF PREFERRED EMBODIMENTS

The invention provides a method for promoting tissue health and repair by modulating the protein profile within the tissue or its surrounding environment. Tissues can become damaged as a result of external forces, such as trauma or injury, which in turn can lead to wounds and/or inflammation. Alternately, tissues can become damaged as a result of internal forces such as disease and genetic factors. Repair of tissue damage is a complex process, which requires control of the environment at the point of damage and the surrounding areas. An aspect of the repair process requires the modulation of the protein profile in and around the damaged tissue. This means that the levels and/or activities of certain proteins must be modulated, i.e., increased or decreased, in order to create an environment that promotes the repair process.

The invention provides compositions that modulate the activity of wound-related proteins such as matrix metalloproteinases (MMPs), cytokines, and growth factors, thereby promoting wound healing. Wound-related proteins include, but are not limited to, MMPs such as MMP-2, MMP-3, MMP-9; TIMPS such as TIMP-1, TIMP-2; TNFα; Interleukins such as IL-β, IL-6, IL-10; Growth Factors such as PDGF-AB, IGF-I, TGFβ, EGF, FGF basic, G-CSF, GM-CSF, VEGF; Interferons such as IFNα, IFNγ; C-reactive protein CRP; and Macrophage Inflammatory Proteins such as MIP-1α, MIP-1β, and MIP2. In general, the compositions of the invention promote wound healing, prevent scarring, improve skin tone and stimulate the development of a smooth, healthy skin.

The invention also provides compositions that modulate the activity of at least one inflammatory or pro-inflammatory protein, thereby preventing or treating inflammation. The inflammation-related proteins include, but are not limited to, TNFα; Interleukins such as IL-β, IL-6, IL-10; serum amyloid A; fibrinogen; Interferons such as IFNα, IFNγ; CRP; Macrophage Inflammatory Proteins such as MIP-1α, MIP-1β, MIP2; and MMPs such as MMP-2, MMP-3, and MMP-9;. Furthermore, since wound healing and inflammation generally go hand-in-hand, the wound-related proteins listed above play a role in inflammation. Thus, many wound-related proteins are also included in the category of inflammation-related proteins.

The term “wound” as used herein, refers to a tissue lesion or area of destruction caused by external factors or the presence of an underlying physiological disorder. The wounds may be localized or cover a large area of skin and tissue surface, and may either be open or have intact skin or tissues covering the area. Wounds or damage tissue may be cutaneous in nature, but may also be found in other tissues throughout the body. The external factors that cause dermatologic wounds to essentially develop are commonly irradiation, mechanical, thermal or chemical trauma. As a consequence of their formation, tissue lesions lead to blood and fluid loss and decreased function, while disruption of the protective function of the skin could allow pathogens, foreign bodies and toxins to enter the body.

According to the invention, a composition comprising a mixture of proteases is useful for the treatment of wounds and skin conditions such as inflammation. Administration of such a mixture modulates the activity of wound-related proteins, and diminishes the rate of tissue destruction, inflammation, edema, fever, pain, itching, and hyperpermiability of endothelium in wounds. Hence, such a protease mixture can provide an improvement in wound healing. Additionally, the administration of such a mixture degrades inflammation-related proteins, and diminishes the intensity of inflammation in skin or wounds. Hence, such a protease mixture can improve the wound healing process by providing a faster rate of resolution to inflammation as well as decrease scarring.

An embodiment of the invention provides compositions that are useful for the management of the environment in and around pre-cancerous and cancerous cells. These cells secrete enzymes, cytokines and growth factors in order to evade the immune system and to establish a blood supply. The compositions of the invention can be used to modulate the microenvironment of the pre-cancerous and cancerous cells in a subject, thereby promoting a normal environment and diminishing the ability of these cells to establish a permanent foothold at their location by thwarting their manipulative and subversive use of MMPs and certain cytokines and growth factors such as FGF basic, VEGF, PDEGF, Ang2, and EphrinB2. If the microenvironment returns to normal, the pre-cancerous and cancerous cells can fall prey to the immune system and lack of nutrients, but without the adverse side effects of chemotherapy, thereby promoting healing and improved health.

Most protein modulation strategies involve preventing activity of the respective proteins with organic small molecules. These compounds are often toxic to the body and are not naturally occurring molecules. Use of natural polypeptides such as proteases to modulate protein levels and activity provides a high degree of proteinase control without toxic side effects. Unlike small molecule inhibition strategies, the protease mixtures of the invention can be used to degrade specific proteins such as MMPs, while leaving growth factors and other beneficial polypeptides intact. The protease mixture can be freely introduced onto the skin, into the wound environment, or can be tethered to, or delivered by, an appropriate carrier or vehicle depending on the wound.

The invention provides a high degree of control over the level of wound-related and inflammation-related protein activity for healing chronic wounds. For example, as some amount of MMP level is required during chronic wound healing, one of skill in the art may choose to only partially inhibit the activity of one or more MMPs. By varying the type and amount of proteases applied, the degree of protein degradation (such as MMP degradation), and consequently inhibition, can be controlled.

One of skill in the art can choose an appropriate protease or combination of proteases to achieve the quality and quantity of modulation desired using available teachings in combination with the teachings provided herein. As used herein, the term “modulation” refers to the variation of the native activity or levels of a protein. Thus, the process of modulation can involve inhibition of a particular protein's activity via degradation or other means. Alternately, modulation of a protein's activity can take the form of an activation step, for e.g., the activation of a pro-enzyme to its active enzymatic form via degradation or other means. “Quality” of inhibition or activation refers to the type of protein targeted. For example, different MMPs can have somewhat different substrates and sites of activity. “Quantity” of inhibition or activation refers to the overall amount of inhibition or activation from all proteins that are targeted by the protease mixture. The type and quantity of protease(s) used determines the level of inhibitory and/or activation modulatory effects on the target protein(s). One of skill in the art can readily make modifications to the protease mixtures provided by the invention and observe the type and degree to which a given protein, such as, for example, a MMP is inhibited.

According to the invention, a mixture of proteases that is useful for wound healing, reducing inflammation and promoting development of healthy skin is provided. As provided herein, the term “protease” is used synonymously with the terms “proteinase” and “peptidase.” The protease mixtures provided by the invention inhibit the activity of many types of matrix metalloproteinases, primarily by degradation of the MMPs. Moreover, the protease mixture can be adjusted so that it inhibits a broad spectrum of metalloproteinases. Alternately, the mixture can be modified so that only one or a few select metalloproteinases are inhibited. The protease mixture of the invention can inhibit the activity of many types of matrix metalloproteinases. The protease mixture of the invention can also prevent the activation of proenzyme matrix metalloproteinases, as well as inhibit the enzymatic activity of mature matrix metalloproteinases.

In certain embodiments of the invention the protease mixture can be changed so that certain proteins, including MMPs, are activated. In certain types of activation, the pro-form of a protein is activated to form the mature form of the protein. Such an activation process provides an active protein that is capable of participating in the wound healing process. An example of this type of activation is the use of proteases to activate specific MMPs to modulate the wound environment of wounds displaying keloids or exuberant granulation tissue formation. In these types of wounds or scars, excessive amounts of ECM collagen, and granulation tissue are deposited. The amount can be so great that the wound cannot close or may form so much excessive tissue; it appears as a tumor protrudance. These types of wounds and scars are a result of a dysfunctional micro-environment in which too few MMPs are active, fibroblast secrete collagen unregulated, and/or cytokine and growth factors are depressed (i.e. IFNγ) or expressed in excess (i.e. TNFα, IL-6). Application of an embodiment of the invention (with or without surgical intervention) could modulate the micro-environment to promote a return to normal wound healing or normal scar remodeling.

The protease mixtures provided by the invention may inhibit the activity of many types of proteins, primarily by degradation. An embodiment of the invention provides a protease mixture that is capable of broadly inhibiting a large number of different proteins. Another embodiment of the invention provides a protease mixture that inhibits either a single protein or a selected few proteins. A further embodiment of the invention provides a protease mixture that activates one or more proteins. The activation of the protein occurs via cleavage of a dormant or less-active form, which provides an active form of the protein. The protease mixture of the invention can modulate the activity of many types of proteins. The protease mixture of the invention can also prevent the activation of pro-forms of protein molecules, as well as inhibit the enzymatic activity of mature forms of protein molecules. Another embodiment of the invention provides a protease mixture that inhibits one or more protein(s) and activates one or more different protein(s).

According to an embodiment of the invention, a protease mixture can selectively degrade certain proteins such as MMPs and/or inflammation-related proteins at the site of the wound, while beneficial proteins such as TIMP-1 and PDGF are spared from degradation, i.e., certain proteins are resistant to degradation, while others undergo proteolytic degradation.

The proteolytic activity of a protease can be assessed by any procedure available to one of skill in the art. Many different assay procedures are available to determine whether or not a particular protease or mixture of proteases exhibit proteolytic activity. One such technique is an ELISA assay.

According to the invention, the protease mixture comprises at least one protease. The protease mixture comprises at least one hydrolase enzyme such as aminopeptidase, aspartic endopeptidase, cysteine endopeptidase, cysteine-type carboxypeptidase, dipeptidase, dipeptidyl-peptidase, metallocarboxypeptidase, metalloendopeptidase, omega peptidase, peptidyl-dipeptidase, serine endopeptidase, serine-type carboxypeptidase, tripeptidyl-peptidase, and/or threonine endopeptidase families.

Examples of proteases include, but are not limited to, acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, and thrombin.

For example, the use of bacterial leucyl aminopeptidase results in the release of an N-terminal amino acid, thus inactivating the certain target molecule functions. Another example of a protease of the invention, the use of complement subcomponent C1r protease selectively cleaves the bond in complement subcomponent C1s to activate form of C1s, which then can activate C2 and C4. Yet another example of a protease of the invention involves the use of fragilysin, which hydrolyzes a variety of bonds of extracellular matrix proteins.

Other conditions which may be treated or prevented by the instant compositions include, but are not limited to, inflammatory diseases. Inflammatory diseases which may be treated or prevented include, for example, septic shock, septicemia, and adult respiratory distress syndrome. Target autoimmune diseases include, for example, rheumatoid arthritis, systemic lupus erythematosus, scleroderma, chronic thyroiditis, Graves' disease, autoimmune gastritis, insulin-dependent diabetes mellitus, autoimmune hemolytic anemia, autoimmune neutropenia, thrombocytopenia, chronic active hepatitis, myasthenia gravis and multiple sclerosis. Target neurodegenerative diseases include, for example, amyotrophic lateral sclerosis, Alzheimer's disease, Parkinson's disease, and primary lateral sclerosis. Target diseases associated with harmful, apoptosis, in other words, those associated with ischemic injury, includes myocardial infarction, stroke, and ischemic kidney disease. The pharmaceutical compositions of this invention may also be used to treat infectious diseases, especially those involved with microbial, parasitic and viral infections.

Further, other inflammation inducing conditions may be treated to ameliorate symptoms associated with inflammation or to diminish the existing inflammation. Inflammation or irritation associated therewith may be from a variety of sources either physical or chemical as noted above, and may include: insect bites or stings, contact with a particular type plant (e.g., poison oak, etc.), radiation (e.g., U.V.), non-infectious conjunctivitis, ophthalmic injuries, tonsillitis, hemorrhoids (acute), abrasions, ingrown finger or toenail (granulation), skin graft donor sites, vaginitis, dermatitis, psoriasis, herpes simplex (cold sores, aphthous ulcers), pruritis ani/cruri, chemical inflammation, cystic fibrosis, and the like. Moreover, such inflammation or other activities of the MMP family of proteases may lead to lack of elasticity or diminished skin appearance and texture or decreased tissue function. Accordingly, the compositions and methods set forth herein, find utility not only in treating inflammatory diseases, but also for in treatment of the associated conditions and symptoms.

Inflammation is the result of extraneously or intrinsically induced damage to cells or tissue. Such damage may be induced by chemical and/or physical influences upon the skin or mucus membranes of humans and animals. Examples of physical influences are infarction, heat, cold, radiation and electrical shock, and examples of chemical influences are contact with acids, bases and allergens. Inflammation may be induced by microorganisms acting on the skin, as well as being the result of microorganisms invading the human or animal body.

A variety of symptoms are associated with inflammation and include, but are not limited to one or more of the following: pain, increased surface temperature, heat, redness, whelps, hives, edema, swelling, itching, pruritus, pain, and reduced or ceased function. The inflammatory responses that may be ameliorated may be on the skin or a mucus membrane of a human or animal, such as a mammal, and includes, but is not limited to, conditions such as inflammation around erupting wisdom teeth, following extraction of teeth, periodontal abscesses, prosthesis induced pressure sores on the mucosa, fungal infections, for treating exposed bone surface in alveolitis sicca dolorosa, which is a painful condition which may arise following extraction of teeth, chronic and acute inflammatory diseases including, but not limited to, pancreatitis, rheumatoid arthritis, osteoarthritis, asthma, inflammatory bowel disease, and psoriasis. Several morphological changes, including a decreased moisture content of the stratum comeum, coupled with reduced eccrine and sebaceous gland output can decrease the presence of these components which protect the skin and allow for loss of collagen, the major skin protein. These morphological changes which result in a loss of integrity of the horny layer of the skin can be caused by a variety of conditions. Among such conditions are environmental, e.g., sun or wind exposure, trauma or wounds, e.g., cuts, burns or abrasions, exposure to chemicals such as alkaline soaps, detergents, liquid solvents, oils, preservatives, and disease, e.g., eczema, psoriasis, seborrheic dermatitis. Accordingly, compositions and methods that suppress the protease activity of the MMP family of proteases are useful in maintaining the skin.

Proteases of the invention can be used to heal wounds and are particularly beneficial for chronic wound healing. Individual proteases, protease variants, polypeptide derivatives and mixtures thereof (e.g. those with different sequences) can be combined in a formulation to promote wound healing and to prevent or treat skin problems. Optimal healing and skin regeneration may require some matrix metalloproteinase activity. Hence, the compositions and formulations of the present invention do not necessarily promote maximal inhibition of matrix metalloproteinases. Instead, the activity of the polypeptide inhibitor formulation is varied as needed to optimize healing and promote healthy skin development. Lesser or greater levels of inhibition can be achieved by varying the type, content and amount of inhibitor polypeptides so that healing and healthy skin development is promoted. Depending on the wound etiology, the patient immune system and the tissue trauma, various formulations of the invention could be developed in order to provide an optimal protein and enzyme activation and inactivation ratios specific for the disease.

To promote healthy skin development and/or treat wounds, proteases of the invention are introduced onto the skin or tissues or into wounds in any manner chosen by one of skill in the art. For example, proteases can be formulated into a therapeutic composition containing a therapeutically effective amount of one or more proteases and a pharmaceutical carrier. Such a composition can be introduced onto skin or into the wound as a cream, spray, foam, gel, solution or in any other form or formulation. In another embodiment, proteases of the invention can be formulated into a skin covering or dressing containing a therapeutically effective amount of one or more proteases impregnated into, covalently attached or otherwise associated with a covering or dressing material. In one embodiment, the skin covering or dressing permits release of the protease. Release of the protease can be in an uncontrolled or a controlled manner. Hence, the skin coverings or wound dressings of the invention can provide slow or timed release of the protease into a wound. Skin coverings and dressing materials can be any material used in the art including, but not limited to bandage, gauze, sterile wrapping, hydrogel, hydrocolloid and similar materials.

A therapeutically effective amount of a protease of the invention is an amount of protease that modulates the target protein activity or levels, such as a matrix metalloproteinase, to a degree needed to promote healthy tissue development and/or wound healing. For example, when present in a therapeutic or pharmaceutical composition, the amount of proteases of the invention can be in the range of about 0.001% to about 35% by weight of the composition. The proteases can form about 0.5% to about 20% by weight of the composition. Alternately, the proteases form about 1.0% to about 10% by weight of the composition. The therapeutically effective amount of protease necessarily varies with the route of administration. However, the amount of the protease required for healthy skin development or wound treatment will vary not only with the route of administration, but also the nature of the condition being treated and the age and condition of the patient and will be ultimately at the discretion of the attendant physician or clinician. The dosage and method of administration can also vary depending upon the location of the skin or tissue to be treated and/or upon severity of the wound.

The protease mixtures of the invention can be formulated as pharmaceutical compositions and administered to a mammalian host, such as a human patient in a variety of dosage forms adapted to the chosen route of administration, i.e., orally or parenterally, by intravenous, intramuscular, inhalation, topical or subcutaneous routes. Thus, the proteases may be systemically administered, for example, intravenously or intraperitoneally by infusion or injection. Solutions of the protease mixture can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion or topical application can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In some cases, one of skill in the art may choose to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the protease or protease conjugate in the required amount in the appropriate solvent with various of the other ingredients enumerated above, as required, followed by sterilization. In the case of sterile powders for the preparation of sterile injectable solutions, methods of preparation include vacuum drying and the freeze-drying techniques, which yield a powder of the active ingredient plus any additional desired ingredient present in the previously sterile solutions.

In some instances, the protease mixture(s) can also be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the proteases may be combined with one or more excipients and used in the form of ingestible tablets, buccal tablets, troches, capsules, elixirs, suspensions, syrups, wafers, and the like. Such compositions and preparations should contain at least 0.1% by weight of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the polypeptide inhibitor may be incorporated into sustained-release preparations and devices.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In general, the protease mixtures of the invention are administered topically for wound treatment and for promoting healthy skin development. The active polypeptides may be administered topically by any means either directly or indirectly to the selected tissue as sprays, foams, powders, creams, jellies, pastes, suppositories or solutions. The term paste used in this document should be taken to include creams and other viscous spreadable compositions such as are often applied directly to the skin or spread onto a bandage or dressing. The protease mixture of the invention can be covalently attached, stably adsorbed or otherwise applied to a skin covering or wound dressing material. To facilitate healing after surgery, the active proteases of the invention can be applied directly to target tissues or to prosthetic devices or implantable sustained released devices. The compositions can be administered by aerosol, as a foam or as a mist, or gel or solution, with or without other agents, directly onto the skin or wound.

The proteases can be administered in a formulation that can include an emulsion of the protease in a wax, oil, an emulsifier, water, and/or a substantially water-insoluble material that forms a gel in the presence of water. The formulation provides the desirable properties of an emulsion, in that it is spreadable and has the creamy consistency of an emulsion, yet that does not break down when subjected to normal sterilization procedures, e.g. steam sterilization, because the gel stabilizes the emulsion. It also exhibits better water retention properties than a conventional gel because water is held both in the emulsion and in the gel.

The formulation can also contain a humectant to reduce the partial vapor pressure of the water in the cream or lotion to reduce the rate at which the cream or lotion dries out. Suitable humectants are miscible with water to a large extent and are generally suitable for application to the skin. Polyols are especially suitable for the purpose and suitable polyols may include monopropylene glycol or glycerin (glycerol). The polyol may be present in proportions of 20 50% (by weight) of the total formulation; alternatively the range is 30 40%. This relatively high proportion of polyol also ensures that if the paste should dry out to any degree, the resulting paste remains soft and flexible because the glycerin may act as a plasticiser for the polymer. When the paste is applied on a bandage, for example, it may therefore still be removed easily from the skin when the paste has lost water without the need to cut the bandage off. The polyol also has the advantage of functioning to prevent the proliferation of bacteria in the paste when it is in contact with the skin or wound, particularly infected wounds.

The formulation can include other ingredients such as antibacterial agents, antifungal agents, anti-inflammatory agents, and the like. Other ingredients may also be found suitable for incorporation into the formulation such as vitamins and herbal agents.

An example of a wax for the emulsion is glyceryl monostearate, or a combination of glyceryl monostearate and PEG100 stearate that is available commercially as CITHROL GMS/AS/NA from Croda Universal Ltd. This combination provides both a wax and an emulsifier (PEG 100 stearate) that is especially compatible with the wax, for forming an emulsion in water. A second emulsifier can be included in the formulation to increase the stability of the emulsion, for example, a PEG20 stearate, such as CITHROL 1OMS that is supplied by Croda Universal Ltd. The total concentration of emulsifier in the cream should normally be in the range of from 3 15%. Where two emulsifiers are used, one may be present in a greater concentration than the other.

The water-insoluble material forms a gel with the water of the formulation. The material is therefore hydrophilic but does not dissolve in water to any great extent. The material can be a polymeric material, for example, a water-absorbing non water-soluble polymer. However, non-polymeric materials that form gels with water and that are stable at elevated temperatures could also be used, e.g. clays such as kaolin or bentonite. Some polymers used in the invention are super-absorbent polymers that comprise hydrophilic cellulose derivatives that have been partially cross-linked to form a three dimensional structure. Suitable cross-linked cellulose derivatives include those of the hydroxy lower alkyl celluloses, wherein the alkyl group contains from 1 to 6 carbon atoms, e.g. hydroxyethyl cellulose or hydroxypropylcellulose, or the carboxy-celluloses e.g. carboxymethyl hydroxyethyl cellulose or carboxy methylcellulose. An example of a polymer that may be used in the invention is a partially cross-linked sodium carboxy methylcellulose polymer supplied as AKUCELL X181 by Akzo Chemicals B.V. This polymer is a superabsorbent polymer in that it may absorb at least ten times its own weight of water. The cross-linked structure of the polymer prevents it from dissolving in water but water is easily absorbed into and held within the three-dimensional structure of the polymer to form a gel. Water is lost less rapidly from such a gel than from a solution and this is advantageous in slowing or preventing the drying out of the cream formulation. The polymer content of the formulation is normally less than 10%, for example, the polymer content can range from about 0.5 to about 5.0% by weight, or from about 1.0% to about 2% by weight.

The formulation may be sterilized and components of the formulation should be selected, by varying the polymer content, to provide the desired flow properties of the finished product. That is, if the product to be sterilized, then the formulation should be chosen to give a product of relatively high viscosity/elasticity before sterilization. If certain components of the formulation are not to be sterilized, the formulation can be sterilized before addition of those components, or each component can be sterilized separately. The formulation can then be made by mixing each of the sterilized ingredients under sterile conditions. When components are separately sterilized and then mixed together, the polymer content can be adjusted to give a product having the desired flow properties of the finished product. The emulsion content determines the handling properties and feel of the formulation, higher emulsion content leading to increased spreadability and creaminess. Sterilization by irradiation by those skilled in the art does not lead to a decrease in activity of the protease(s).

The formulation may be packaged into tubes, tubs or other suitable forms of containers for storage or it may be spread onto a substrate and then subsequently packaged. Suitable substrates include dressings, including film dressings, and bandages.

Because of their diverse applicability, the compositions of the invention are suitable for use as medicines, cosmetics, prescription drugs and over-the-counter (OTC) medications.

### WORKING EXAMPLES

Test and Control Materials

Elta protease formulation SAP1439 (Elta Proteases) was used as a solution. MMP standard (Sigma, St. Louis, Mo.) was prepared from concentrated active-and pro-MMP-2 and MMP-9 and sterile water. Serial dilutions (1×, 2×, 4×, 8×, 16×) of the sterile protease mix were prepared with sterile water. A uniform stock of chronic wound fluid (CWF) was prepared for the experiments by mixing samples obtained from multiple patients.

Zymography

Sample preparation-MMP standard was incubated (1:1) with each of the Elta Proteases 8× and 16× dilutions for 30 minutes. A 2× dilution of the MMP standard and 2× dilutions of the Elta Proteases dilutions were also prepared for comparison. Overnight and acute incubations of CWF with 1×, 2×, 4×, 8×, and 16× Elta Proteases dilutions were prepared at room temperature and at 37° C., along with 2× dilutions of the 8× Elta Proteases dilution and the CWF standard. Sample buffer (20 μL) was added at the end of the incubation of each sample. Ten minutes later the samples were added to the zymogram gel.

Zymogram-Samples were added to a 10% Zymogram Gel (Invitrogen, Carlsbad, Calif.). The gel was run at a constant 125V at 4° C. After 2 hours, the gel was incubated in renaturing buffer for 30 minutes. The buffer was then replaced with developing buffer. After 30 minutes at room temperature, the gel was placed on a rocker platform set at 7 for overnight incubation at 37° C. The developing buffer was replaced with Coomassie stain (2 ml Rapid Coomassie Stain in 40 ml 7.5% methanol-5.0% acetic acid), and the gel incubated at room temperature on an orbital shaker (70 rpm) for 60 minutes. The stain was replaced with destain (7.5% methanol-5.0% acetic acid) and incubated for 10 minutes on an orbital shaker. Destain was replaced with deionized water, and the gel was photographed with a digital camera.

ELISA Assays

Sample Preparation-Prior to running the ELISA assays, the CWF standard was tested to determine the baseline levels of MMP9, TIMP-1, TNFα, IL-1β, and PDGF. CWF standards were spiked with purified concentrations of 640 pg/ml TNFα and 4000 pg/ml PDGF stock solutions to achieve an adequate baseline concentration. Aliquots were prepared by combining the target protein in a 1:1 ratio with Elta Proteases or PBS control. Aliquots were removed for a time-zero reading. All reactions were incubated at 37° C. and room temperature and additional aliquots removed at 1, 4, 8, and 24 hours. With the exception of the TIMP-1 and IL-1β samples, the aliquots were mixed 10:1 with a general-purpose protease inhibitor (Sigma; St. Louis, Mo.) and frozen at −80° C. TIMP-1 samples were diluted 1:25 in the kit assay buffer prepared with and without the general purpose protease inhibitor (1:100) to determine the effect of Elta Proteases on TIMP-1 in CWF and the TIMP-1 ELISA standards. IL-1β samples were not mixed with a protease inhibitor.

All ELISAs were performed per manufacturer specifications. All samples were run in duplicate wells and all ELISAs repeated at least twice on separate days.

Active MMP9 concentrations were quantified using the Matrix Metalloproteinase-9 (MMP 9) Biotrak Activity Assay System (Amersham; Piscataway, N.J.) per manufacturer instruction. CWF had adequate MMP9 levels and was diluted 150× in ELISA standard diluent before running the ELISA.

TIMP-1 concentration was quantified using the TIMP-1, Human Biotrak™ ELISA (Amersham; Piscataway, N.J.) per manufacturer instruction except the TIMP-1 standards were prepared with and without a general purpose protease inhibitor (Sigma; St. Louis, Mo.) diluted 1:100 in the kit assay buffer.

TNFα concentration was quantified using the Tumour Necrosis Factor Alpha [(h)TNFα] Human Biotrak ELISA System (Amersham; Piscataway, N.J.). CWF with TNF-α added was run undiluted.

IL-1β concentrations were quantified using the Quantikinee human IL1-β ELISA (R&D Systems, DLB50) per manufacturer instruction. No protease inhibitor was added before running the ELISA. The CWF had adequate levels of IL-1β, so no exogenous protein was added. The reactions in CWF were diluted 100× in water.

PDGF-AB concentrations were quantified using the Quantikine® human PDGF-AB ELISA (R&D Systems, DHD00B) per manufacturer instruction. CWF was diluted 2×.

ELISA Results

Active MMP9 concentrations in CWF were assessed using ELISA. Complete degradation and complete inactivation of active MMP9 occurred within the first hour of incubation at 37° C. with the Elta Proteases and within 8 hours at room temperature, see Table 1 (Percent reduction by time and temperatures for various proteins by ELISA). Controls of CWF alone had a slight degradation of active MMP9 over time regardless of incubation temperature.

TIMP-1 concentrations in CWF were assessed using ELISA. Prior to initiating the ELISA, TIMP-1 standard assay buffer with and without a general-purpose protease inhibitor were tested and compared. Spectrophotometrical absorbance readings were higher for the standards containing inhibitor than standards that were not exposed to the inhibitor suggesting TIMP-1 was being degraded during the 2-hour room temperature incubation period. Also, observed was TIMP-1 standards degraded slightly in the assay buffer over time. To assess the effect of the Elta Proteases on TIMP-1 concentrations in CWF, samples were incubated and assayed by ELISA. At 24 hours, the decrease of TIMP-1 levels were similar to the control indicating TIMP1 was resistant to degradation of Elta Proteases, see Table 1.

TNFα concentrations in CWF were run undiluted and assayed using ELISA. Proteolysis occurred within the ELISA wells since the protease inhibitor was not added to the sample until after the incubation period. Complete TNFα degradation occurred within 8-10 hours in the presence of Elta Proteases at 3 7° C. and were reduced greater than 90% at room temperature, see Table 1. Comparatively, TNFα levels in the controls were reduced 35% at 37° C. and 2% at room temperature.

IL-1β concentrations in CWF were assessed using ELISA. Proteolysis occurred within the ELISA wells since the protease inhibitor was not added to the samples. At times up to 24 hours, the levels of IL-1β exposed to Elta Proteases were similar compared to controls at both room temperature and 37° C. At both temperatures, the IL-1β levels exposed to the Elta Proteases showed less degradation than the controls, see Table 1. These results suggest the Elta Proteases do not degrade the IL-1β protein in CWF, but may also confer protection to the protein.

PDGF-AB concentrations in CWF were assessed using ELISA. CWF was spiked with exogenous PDGF-AB to determine the affects of the Elta Proteases on the protein. At times up to 24 hours, the levels of PDGF exposed to Elta Proteases were similar compared to controls at both room temperature and 37° C. Although the PDGF concentrations were above natural physiological levels, significant proteolysis of PDGF was not observed, suggesting resistance to degradation. At both temperatures, the PDGF levels exposed to the Elta Proteases showed less degradation than the controls, see Table 1. These results suggest the Elta Proteases do not degrade the PDGF protein in CWF, but may also confer protection to the protein.

The ELISAs showed interesting and surprising results. The Elta Proteases were able to degrade active MMP9 and TNFα at room temperature, but more markedly at body temperature. Rapid and complete MMP degradation occurred within 1 hour and within 8-10 hours for TNFα in CWF from patient samples. Unlike MMP and TNFα, TIMP, IL1β, and PDGF were not degraded by the Elta Proteases during the 24 hour incubation period, even at 37° C. Even more interesting was the observation the controls had more proteolysis of target proteins IL-1β and PDGF by CWF than in samples incubated with the CWF and the Elta Proteases.

TABLE 1

| Time & | Test | CWF | CWF | CWF | CWF | CWF |
|---|---|---|---|---|---|---|
| Temp | Article | MMP9 | TIMP | TNFα | IL1β | PDGFAB |
| Time 0 | Control | 0% | 0% | 0% | 0% | 0% |
| RT | Proteases | 28% | 36% | 56% | −33% | 2% |
| Time 1 | Control | 6% | 26% | 5% | −22% | −2% |
| RT | Proteases | 54% | 44% | 58% | −35% | −3% |
| Time 4 | Control | 30% | 45% | 7% | −21% | 0% |
| RT | Proteases | 90% | 53% | 59% | −36% | −2% |
| Time 8 | Control | 17% | 57% | 9% | −34% | 13% |
| RT | Proteases | 100% | 60% | 80% | −59% | −5% |
| Time 24 | Control | 48% | 74% | 2% | −26% | 19% |
| RT | Proteases | 100% | 75% | 96% | −26% | −8% |
| Time 0 | Control | 0% | Not Done | 0% | 0% | 0% |
| 37° | Proteases | 15% | Not Done | 56% | 0% | 10% |
| Time 1 | Control | −12% | Not Done | 2% | 13% | 13% |
| 37° | Proteases | 100% | Not Done | 66% | −11% | 8% |
| Time 4 | Control | 10% | Not Done | 5% | 3% | 10% |
| 37° | Proteases | 100% | Not Done | 93% | 14% | 9% |
| Time 8 | Control | 40% | Not Done | 15% | 27% | 11% |
| 37° | Proteases | 100% | Not Done | 99% | 11% | 12% |
| Time 24 | Control | 52% | Not Done | 35% | 27% | 29% |
| 37° | Proteases | 100% | Not Done | 100% | 15% | 33% |

Zymography

The ability of Elta Proteases to degrade purified active and pro forms of MMP 2 and 9 standards and gelatinases in pooled chronic wound fluid (CWF), was assayed using zymogram gels. Initial experiments revealed Elta Proteases degraded the gelatin contained within the zymogram gel. Serial dilutions of Elta Proteases (2×, 4×, 8×, 16×) were tested to detect the optimal dilutions to run on the zymogram gels. The 8× and 16× dilutions had the least amount of background degradation while allowing for reactions within the CWF to be observed.

Purified active and pro forms of MMP 2 and 9 standards were incubated with the 8× and 16× dilutions of Elta Proteases. All of the MMP standards were completely degraded by both dilutions except the active MMP2 that was incubated for 30 minutes at room temperature. Molecule weight bands for 180, 92, 86, 72, and 66 kDa were degraded equally well.

Gelatinases in pooled CWF were then incubated with the 8× dilution of Elta Proteases. Degradation of the CWF gelatinases by the 8× dilution was noticeable after only 30 minutes of incubation. Degradation was even more pronounced after 24 hours of incubation, especially in samples containing less diluted Elta Proteases. While all molecule weight bands were degraded, the bands for 92, 72, and 66 kDa were degraded better than the 180 and 86 bands.

The zymograms clearly demonstrated the ability of the Elta Proteases to degrade MMP standards and CWF gelatinases, even when diluted. Increased incubation temperature and time both enhanced the ability of Elta Proteases to degrade the MMPs and CWF gelatinases resulting in inactivation. An increase in Elta Protease concentration also improved the rate of degradation compared to diluted samples. These results confirmed the ELISA results previously discussed.

What is claimed is:

1. A composition comprising at least one protease; and optionally a pharmaceutically acceptable carrier, diluent or excipient, wherein the protease can modulates the action or level of at least one protein in a physiological environment.

2. The composition of claim 1, wherein said protein is a wound-related protein or an inflammation-related protein.

3. The composition of claim 1, wherein said physiological environment is a tissue.

4. The composition of claim 1, wherein said protease is selected from one or more of aminopeptidase, aspartic endopeptidase, cysteine endopeptidase, cysteine-type carboxypeptidase, dipeptidase, dipeptidyl-peptidase, metallocarboxypeptidase, metalloendopeptidase, omega peptidase, peptidyl-dipeptidase, serine endopeptidase, serine-type carboxypeptidase, tripeptidyl-peptidase, threonine endopeptidase or active variants, homologues, derivatives or fragments thereof.

5. The composition of claim 2, wherein said wound-related protein or inflammation-related protein is present in a damaged tissue.

6. The composition of claim 2, wherein said wound-related protein or inflammation-related protein is present in or near cancerous or pre-cancerous cells.

7. The composition of claim 5, wherein said damaged tissue is a wound.

8. The composition of claim 7, wherein said wound is an acute wound or a chronic wound.

9. The composition of claim 5, wherein said damaged tissue is an ulcer.

10. The composition of claim 2, further comprising additional ingredients.

11. The composition as defined in any one of claims 1 to 10 for use in medicine, cosmetics or prescription drugs.

12. Use of a composition as defined in any one of claims 1 to 10 in the manufacture of a pharmaceutical to treat damaged tissue, such as wounds.

13. Use of a composition as defined in any one of claims 1 to 10 in the manufacture of a pharmaceutical to treat ulcers.

14. A method of therapy, said method comprising administering to a subject a composition as defined in any one of claims 1 to 10 and in an amount to treat damaged tissue.

15. A method according to claim 14 wherein said damaged tissue is the result of a wound.

16. A method according to claim 14 wherein said damaged tissue is the result of an ulcer.

17. A method according to claim 14 wherein said damaged tissue is the result of a cancer.

18. A process for preparing a composition as defined in any one of claims 1 to 10; said process comprising the steps of: (i) performing an assay to identify one or more agents that are capable of acting as a protease as defined in any one of claims 1 to 10; (ii) admixing one or more of said agent(s) with a pharmaceutically acceptable carrier, diluent or excipient.

19. A process according to claim 18 wherein said process also includes the subsequent step of: (iii) administering said composition to a subject in need of same.

20. A process for preparing a pharmaceutical for use in treating damaged tissue, the process comprising forming a composition by admixing (a) at least one protease with (b) a pharmaceutically acceptable carrier, diluent or excipient; wherein the protease can modulate the action of at least one protein, wherein said protein is a wound-related protein or an inflammation-related protein.

21. Use of a composition as defined in any one of claims 1 to 10 in the manufacture of a pharmaceutical to treat a subject that is being treated with a protease as defined in any one of claims 1 to 10.

22. A method of therapy, said method comprising administering to a subject a composition as defined in any one of claims 1 to 10 and in an amount to treat damaged tissue, wherein all or some of said protease as defined in any one of claims 1 to 10 is administered topically, orally, parenterally, intravenously, intramuscularly, by inhalation, subcutaneously, intraperitoneally, by infusion or by injection.

23. A method for repairing damaged tissue comprising,
identifying an area of tissue damage in a subject; and
administering a composition comprising at least one protease to the area of tissue damage.
